(19) United States
(12) Patent Application Publication
(10) Pub. No.: US 2022/0235328 A1
(43) Pub. Date: Jul. 28, 2022

(54) HUMAN DISEASED AND NORMAL KIDNEY EPITHELIAL CELL CULTURES AND USES THEREOF

(71) Applicant: DISCOVERYBIOMED, INC., Birmingham, AL (US)

(72) Inventors: Erik Schwiebert, Birmingham, AL (US); Deborah Mai, Birmingham, AL (US); Matthew J. Redmann, Hoover, AL (US)

(21) Appl. No.: 17/615,419

(22) PCT Filed: May 28, 2020
(86) PCT No.: PCT/US2020/034992

§ 371 Date: Nov. 30, 2021

Related U.S. Application Data
(60) Provisional application No. 62/854,756, filed on May 30, 2019.

(51) Int. Cl. C12N 5/071

(52) U.S. Cl. CPC C12N 5/0686; C12N 2510/04; C12Q 1/6869; C12N 2533/90; C12N 2513/00

(57) ABSTRACT

Provided herein are compositions and methods for producing immortalized, cystogenic, single cyst-derived cells from a polycystic kidney. Populations of immortalized, cystogenic, single cyst-derived cells from a polycystic kidney are also provided.

This application claims the benefit of U.S. Provisional Application No. 62/854,756 filed on May 30, 2019, which is hereby incorporated by reference in its entirety.

### BACKGROUND

Poly cystic kidney disease (PKD) is characterized by distribution of a large number of cysts in significantly enlarged kidneys. These cysts cause impairment of kidney function and can eventually cause kidney failure. In humans, PKD can be inherited in autosomal dominant (ADPKD) or autosomal recessive (ARPKD) forms. ADPKD is the most common dominantly inherited kidney disease in humans and the leading genetic cause of eventual dialysis or transplantation. In ADPKD, fluid-filled cysts develop and enlarge in both kidneys, eventually leading to kidney failure. ADPKD is the fourth leading cause of kidney failure and more than fifty percent of people with ADPKD will develop kidney failure by age 50. Once a person has kidney failure, dialysis or a kidney transplant are the only options for survival. Currently, the field lacks a practical solution for identifying agents that can treat or prevent PKD.

### SUMMARY

Provided herein is a method of producing immortalized, cystogenic, single cyst-derived cells from a polycystic kidney. The method includes the steps of (a) isolating one or more cells from a single cyst in a polycystic kidney; (b) culturing the isolated one or more cells in a culture dish or well under conditions for expanding the one or more cells to create a plurality of single cyst-derived cells; (c) transferring the plurality of single-cyst derived cells to a three dimensional culture system configured for cyst formation; (d) optionally, transferring the plurality of single-cyst derived cells to a three dimensional culture system configured for cyst formation; (e) determining whether the plurality of single-cyst derived cells form cyst-like structures in the three dimensional culture; and (f) immortalizing one or more of the plurality of single-cyst derived cells that form cyst-like structures.

Further provided is a population of immortalized, cystogenic, single cyst-derived cells from a polycystic kidney. Also provided is a three dimensional culture of immortalized, cystogenic, single cyst-derived cells from a polycystic kidney comprising (a) a population of immortalized, cystogenic, single cyst-derived cells and (b) a three-dimensional gel.

Further provided is a method of screening for an agent that prevents or treats polycystic kidney disease. The method includes the steps of (a) contacting a culture of cystogenic, single cyst-derived cells from a polycystic kidney with an agent to be screened; (b) transferring the contacted cells to a three dimensional culture system configured for cyst formation; and (c) determining the level of cyst-like formations in the three dimensional culture system of the contacted cells after treatment with the agent as compared to a control level. A reduced level of cyst-like formations indicates the agent prevents or treats polycystic kidney disease.

### DESCRIPTION OF THE DRAWINGS

The present application includes the following figures. The figures are intended to illustrate certain embodiments and/or features of the compositions and methods and to supplement any description(s) of the compositions and methods. The figures do not limit the scope of the compositions and methods, unless the written description expressly indicates that such is the case.

FIG. 1 shows a schematic of how remnant human ADPKD diseased kidney tissues are processed into single cyst cultures in primary culture.

FIG. 2 shows a primary human ADPKD (huADPKD) single cyst culture after plating cells isolated from a single cyst in a polycystic kidney, but before the cells were washed.

FIG. 3 shows a primary huADPKD single cyst culture after washings.

FIG. 4 shows a primary huADPKD single cyst culture well that was confluent after incubation for nine days.

FIG. 5 shows the effects of tolvaptan at various concentrations and timepoints, on small cysts formed by primary huADPKD cells, in a three-dimensional culture system in cyst prevention assays.

FIG. 6 shows the effects of rapamycin at various concentrations and timepoints, on small cysts formed by primary huADPKD cells, in a three-dimensional culture system in cyst prevention assays.

FIG. 7 shows the effects of bosutinib at various concentrations and timepoints, on small cysts formed by primary huADPKD cells in a three-dimensional culture system in cyst prevention assays.

FIG. 8A and FIG. 8B show the DMSO equivalent vehicle controls on small cysts formed by primary huADPKD cells in a three-dimensional culture system in cyst prevention assays. FIG. 8A shows the results for 1-300 μM DMSO concentration equivalents. FIG. 8B shows the results for 1-30 nM DMSO concentration equivalents in cyst prevention assays.

FIG. 9 shows the effects of tolvaptan in cyst image tracking at a key concentration in cyst attack assays of larger cysts formed by primary huADPKD cells, in a three-dimensional culture system.

FIG. 10 shows the effects of rapamycin in cyst image tracking at a key concentration in cyst attack assays of larger cysts formed by primary huADPKD cells, in a three-dimensional culture system.

FIG. 11 shows the effects of bosutinib in cyst image tracking at a key concentration in cyst attack assays of larger cysts formed by primary huADPKD cells, in a three-dimensional culture system.

FIG. 12 shows the DMSO equivalent vehicle controls on larger cysts formed by primary huADPKD cells in a three-dimensional culture system.

FIG. 13 shows a 384-well microtiter plate design in which the primary huADPKD cells are seeded into a three-dimensional culture system for high-content imaging based cystogenesis bioassays.

FIG. 14 shows examples of high-content imaging of the entire well of a 384-well microtiter plate. Six images are taken to capture the entire well and Z-stacking in layers up through the 3D Biogel are performed. The image is later stitched together to generate the images shown. Cysts expand over time and no stimuli are required to trigger cystogenesis. Cystogenesis occurs in our DBM RenalCyte media.

FIG. 15 shows examples of arginine vasopressin (AVP)-stimulated cystogenesis (bottom row) versus unstimulated controls (top row). The cysts are larger on average in the presence of this physiological cyclic AMP stimulus.

FIG. 16A shows examples of high-content imaging (are labelled with the Ricolinostat concentration used in each well), FIG. 16B shows cell viability analysis, FIG. 16C shows cyst counting, and FIG. 16D shows cyst size analysis with an industry standard control drug, ricolinostat, in an automated and high-content imaging-driven 384-well cystogenesis platform.

FIGS. 17A and 17B show summary data in cyst prevention assays (FIG. 17A) and cyst attack assays (FIG. 17B) comparing Compound 1 versus tolvaptan at nanomolar concentrations.

FIG. 18 shows typical cyst images in a well of a 384-well plate in cyst prevention assays comparing Compound 1 versus tolvaptan at nanomolar concentrations.

FIGS. 19A and 19B show summary data in cyst prevention assays (FIG. 19A) and cyst attack assays (FIG. 19B) comparing Compound 2 versus tolvaptan at nanomolar concentrations.

FIG. 20 shows typical cyst images in a well of a 384-well plate in cyst prevention assays comparing Compound 2 versus tolvaptan at nanomolar concentrations.

FIGS. 21A and B show summary data in cyst prevention assays (FIG. 21A) and Cyst attack assays (FIG. 21B) comparing a less effective Compound 3 versus tolvaptan at nanomolar concentrations.

FIG. 22 shows typical cyst images in a well of a 384-well plate in cyst prevention assays comparing a less effective Compound 3 versus tolvaptan at nanomolar concentrations.

FIG. 23 shows typical cyst images with tolvaptan at nanomolar concentrations in a 384-well plate in cyst prevention assays.

FIG. 24 shows a data summary of cyst prevention assays in 384-well plates showing efficacy of Compounds 1, 2, 4, and 3 and tolvaptan (from left to right) at nanomolar concentrations.

FIG. 25 shows a data summary of cyst attack assays in 384-well plates showing the efficacy of Compounds 1, 2, and 5 (each at 1 μM), which are CFTR correctors and ENaC inhibitors, versus the Vertex CFTR corrector drugs (each at 10 μM) and tolvaptan (10 μM) on larger cysts formed by primary huADPKD cells in a three-dimensional culture system. Data are shown left to right for media only, DMSO control, VX-809, VX-661, Compound 1, Compound 2, Compound 6, and tolvaptan.

FIG. 26 shows a data summary of cyst reduction assays in 384-well plates showing the efficacy of Compounds 1, 2, and 5, which are CFTR correctors and ENaC inhibitors, versus the Vertex CFTR corrector drugs and tolvaptan on smaller cysts formed by primary huADPKD cells in a three-dimensional culture system.

FIG. 27 shows a data summary of cyst prevention assays in 384 well plates showing the efficacy of Compounds 1, and 5, which are CFTR correctors and ENaC inhibitors, versus the Vertex CFTR corrector drugs and tolvaptan on the 3D Biogels before cysts form from primary huADPKD cells seeded into a three-dimensional culture system.

FIG. 28 shows a schematic of the use of the 3D Biogel-driven cystogenesis assay in 384-well plates for rational and pathophysiologically relevant high-throughput screening for novel human ADPKD cystogenesis and inflammasome inhibitors.

FIG. 29 shows the results of an endpoint CellTiterGlo assay (cell viability) for ADPKD cells grown in a 3D Cyst Prevention Assay format for 13 days, after exposure to concentration-response curves of selected compounds.

FIG. 30 shows the cyst numbers for ADPKD cells grown in a 3D Cyst Prevention Assay format for 13 days, after exposure to concentration-response curves of selected compounds.

FIG. 31 shows cyst size measurements for ADPKD cells grown in a 3D Cyst Prevention Assay format for 13 days, after exposure to concentration-response curves of selected compounds.

FIG. 32 shows representative images of ADPKD cells grown in a 3D Cyst Prevention Assay format for 14 days after exposure to concentration-response curve of selected compounds. Images are shown from a concentration of 0.1 μM where no cytotoxicity was observed for any of the candidate therapeutics.

FIG. 33 shows the results of a multiplex assay measuring cell viability and LDH secretion from ADPKD cells.

### DETAILED DESCRIPTION

PKD is a genetic disorder in which renal tubules become structurally abnormal, resulting in the development and growth of multiple cysts within the kidney. These cysts may begin to develop in utero, in infancy, in childhood, or in adulthood. Cysts are non-functioning tubules filled with fluid, which range in size from microscopic to every large, crushing adjacent normal tubules and eventually rendering the normal tubules non-functional as well. PKD is caused by abnormal genes (PKD1, PKD2 or PKD3) that encode abnormal proteins, which affect tubule development. As used throughout, PKD refers to autosomal dominant polycystic kidney disease (ADPKD) or autosomal recessive polycystic kidney disease (ARPKD),

### Methods of Making Cystogenic Single Cyst-Derived Cells

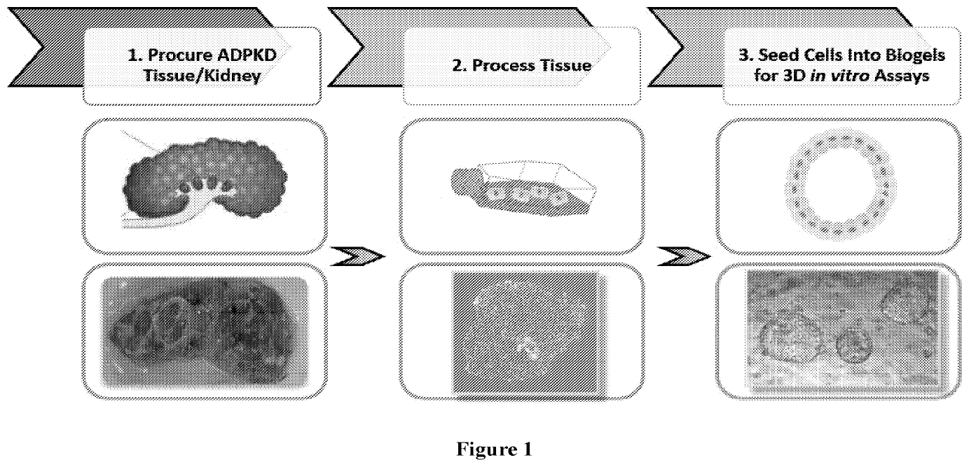

Provided herein are compositions and methods for producing immortalized, cystogenic, single cyst-derived cells from a polycystic kidney. Some methods include the steps of (a) isolating one or more cells from a single cyst in a polycystic kidney; (b) culturing the isolated one or more cells in a culture dish or well under conditions for expanding the one or more cells to create a plurality of single cyst-derived cells; (c) transferring the plurality of single-cyst derived cells to a three dimensional culture system configured for cyst formation; (d) determining whether the plurality of single-cyst derived cells form cyst-like structures in the three dimensional culture; and (e) immortalizing one or more of the plurality of single-cyst derived cells that form cyst-like structures. FIG. 1 shows a schematic of primary processing of single cysts to produce three dimensional cultures useful in the assay systems.

In the methods provided herein, the one or more cells are isolated from a single cyst in a polycystic kidney. In some methods, the polycystic kidney is from a human subject. In some methods, one or more pluralities of single-cyst derived cells can be produced from one or more cells isolated from the same single cyst. In other methods, one or more pluralities of single-cyst derived cells can be produced from one or more cells from different single cysts in a polycystic kidney. Methods of isolating or dissecting single cysts from a polycystic kidney are known in the art. See, for example, Carone et al., “Cyst-derived cells do not exhibit accelerated growth or features of transformed cells in vitro,” Kidney International 35: 1351-1357 (1989). Methods for isolating single cysts are also provided in the Examples.

After isolation, the single cyst or tissue from a single cyst can be further processed. For example, the single cyst or tissue from a single cyst can be further processed using mechanical disruption or enzymatic digestion techniques. (See, for example, Carone et al.). The enzymatic digestion protocol described in the Examples can also be used to isolate one or more cells from the single cyst. The isolated one or more cells are then placed in a culture dish or well and expanded to create a plurality of single cyst-derived cells, i.e., a primary cell culture. The culture dish can be a 35 mm tissue culture plate, a 60 mm tissue culture plate, a 100 mm tissue culture plate, a 150 mm tissue culture plate tissue culture plate, or a larger tissue culture plate. The cells can also be placed in a 6-well, 12-well, 24-well, 48-well, 96-well, 192-well, 384-well or 768-well tissue culture plate. The cells can also be expanded in a flask, for example, in a T-25, a T-75 or a T-175 flask. In any of the methods provided herein, the cells can be expanded until the dish, well, plate, flask etc., is at least about 75%, at least about 80%, at least about 90%, at least about 95%, or at least about 99% confluent.

Optionally, the isolated one or more cells are placed on a permeable filter support-based culture and expanded to create the primary cell culture.

As used throughout, the phrase primary, in the context of a primary cell, refers to a cell that has not been transformed or immortalized. Such primary cells can be cultured, sub-cultured, or passaged a limited number of times (e.g., cultured 0, 1, 2, 3, 4, 5, 6, 7, 8, 9, or 10 times). In some cases, the primary cells are adapted to in vitro culture conditions. In some cases, the primary cells are stimulated or activated.

In some methods, the one or more cells isolated from a single cyst of a polycystic kidney can include one or more fibroblasts. Therefore, during expansion, the plurality of single cyst-derived cells can include fibroblasts. In some examples, the plurality of single cyst-derived cells comprises less than about 10%, less than about 5%, or less than about 1% fibroblasts. Optionally, the one or more cells are cultured in media that maintains the epithelial phenotype of the single cyst-derived cells. Optionally, the one or more cells are cultured in media that maintains the epithelial phenotype of the single cyst-derived cells and increases the proliferation rate of the cells. In some examples, the proliferation rate is increased by at least about 10%, 20%, 30%, 40%, 50%, 60%, 70%, 80%, 90%, 100%, 200%, 300% or 400% as compared to a plurality of cyst-derived cells not contacted with the media.

Once the one or more cells single-cyst derived cells are expanded to create a plurality of single cyst-derived cells, the plurality is transferred to a three dimensional (3D) culture system configured for cyst formation. Optionally, the 3D culture system comprises media that maintains the epithelial phenotype of the single cyst-derived cells and/or increases the proliferation rate of the one or more cells. Since 3D cell culture systems more accurately represent the microenvironment where cells reside in tissues, the behavior of 3D cultured single cyst-derived cells is more reflective of in vivo cellular responses, for example, cyst formation by kidney cells in kidney parenchymal tissue.

While a traditional two-dimensional culture usually grows cells into a monolayer on glass or, more commonly, in tissue culture polystyrene plastic flasks, 3D cell cultures grow cells into 3D aggregates/spheroids using a scaffold/matrix or in a scaffold-free manner. Scaffold/matrix-based 3D cultures can be generated by seeding cells on an acellular 3D matrix or by dispersing cells in a liquid matrix followed by solidification or polymerization. Commonly used scaffold/matrix materials include biologically derived scaffold systems and synthetic-based materials. Commercially available products such as BD Matrigel™ basement membrane matrix (BD Sciences, Franklin Lakes, N.J.), Cultrex® basement membrane extract (Trevigen, Gathersburg, Md.), and hyaluronic acid are commonly used biologically derived matrices. Polyethylene glycol (PEG), polyvinyl alcohol (PVA), polylactide-co-glycolide (PLG), and polycaprolactone (PLA) are common materials used to form synthetic scaffolds. Scaffold-free 3D cell spheroids can be generated in suspensions by the forced floating method, the hanging drop method, or agitation-based approaches. See, for example, Breslin et at, “Three-dimensional cell culture: the missing link in drug discovery,” Drug Discov. Today 18: 240-249 (2013); Gurski et al., “Three-dimensional matrices for anti-cancer drug testing and development,” Oncol. Issues 25: 20-25 (2010); and Edmonson et al., “Three-dimensional cell culture systems and their applications in drug discovery and Cell-Based Biosensors,” Assay Drug Dev. Technol. 12(4): 207-218 (2014). Using 3D cell culture methods, cells grow naturally in a 3D environment, allowing cells to interact with each other, the extracellular matrix (ECM), and their microenvironment. In turn, these interactions in such 3D spatial arrangement affect a range of cellular functions; including cell proliferation, differentiation, morphology; gene and protein expression, and cellular responses to external stimuli.

Optionally, the transferred plurality of single-cyst derived cells have been passaged fewer than five times prior to transfer. Optionally, the transferred plurality of single-cyst derived cells have been passaged fewer than three times prior to transfer. Optionally, the lifetime of any of the primary cultures described herein can be extended by one, two, three, four or more passages, by growing the primary single-cyst derived kidney cells on permeable filter supports prior to seeding the cells into a 3D tissue culture system. In some cases, the cells can be grown in tissue culture plates, wherein each well of the tissue plate contains a clear polyester filter support, which allows visualization of the cells during expansion. In other cases, the cells can be grown on a dish permeable filter support, for example, a support that is made from polycarbonate. Optionally, primary cultures that struggle to form cysts are stimulated with forskolin and/or arginine vasopressin (AVP), which mobilize cyclic AMP (cAMP), or with growth factors like epidermal growth factor (EGF) to trigger cystogenesis in primary cultures that do not form cysts in media alone. In some methods, the 3D tissue culture system includes forskolin. AVP, and/or EGF to enhance cyst formation.

In some methods, the plurality of single-cyst derived cells are transferred to or embedded in a 3-D culture system for an amount of time sufficient to allow cyst formation. For example, the cells can be in the 3-D culture system for one, two, three, four, five, six, seven, eight, nine, ten or more days before and while cysts appear. If cyst formation occurs in the 3D culture system, with or without stimuli, the plurality of cells from a sample of the primary culture, prior to 3D culturing is a cyst-forming plurality that can be immortalized. These immortalized cell lines can be used in assays to identify agents that treat or prevent kidney cyst formation.

In the methods provided herein, one or more cells of the plurality of single-cyst derived cells that form cyst-like structures can be immortalized using methods known in the art. For example, and not to be limiting, the cells can be transfected with a viral oncogene, for example, SV40 and/or a human telomerase (hTERT). See, for example, Sarrab et al., “Establishment of conditionally immortalized human glomerular mesangial cells in culture, with unique migratory properties,”Am. J. Physiol. Renal Physiol. 301(5): F1131-F1138 (2011); Lechner et al., “Human epithelial cells immortalized by SV40 retain differentiation capabilities in an in vitro raft system and maintain viral DNA extrachromosomally,” Virology 185(2): 563-71 (1992); and Lee et al., “Use of exogenous hTERT to immortalize primary human cells,” Cytotechnology 45(1-2): 33-38 (2004)). Other methods involve CDK4 or CDNK2 genetic constructs alone or in combination with the hTERT method of immortalization.

In some methods, one or more cells isolated from a single cyst are genotyped and/or whole exome sequenced. Genotyping allows identification of a primary germline PKD1 mutation(s) that is present in all cells from a single cyst as well as any somatic PKD1 mutations present in one or more cells of the single cyst, Methods for genotyping kidney cells are known in the art and include, but are not limited to, restriction fragment length polymorphism identification (RFLPI) of genomic DNA, random amplified polymorphic detection (RAPD) of genomic DNA, amplified fragment length polymorphism detection (AFLPD), polymerase chain reaction (PCR), DNA sequencing (for example, whole genome or whole exome sequencing), allele specific oligonucleotide (ASO) probes, and hybridization to DNA microarrays or beads.

Examples of germline mutations in PKD1 include, but are not limited to, a P2736R mutation, a L2763V mutation, a M2764T mutation, a frameshift/truncation at E685, a P872X ns/truncation, a R2767 frameshift/truncation, and a G3690E missense mutation. Examples of somatic PKD1 mutations include, but are not limited to, a P3448 frameshift mutation, a A62 frameshift mutation, a L237 frameshift mutation, a L715 frameshift mutation, a Q160 frameshift mutation, a K718 frameshift mutation, a Q542 frameshift mutation, a Y684 frameshift mutation, a N116 frameshift mutation and a G617 frameshift mutation. By genotyping the cells isolated from a single cyst, the genotyped cells can be used to identify agents that treat or prevent cyst formation associated with the specific mutation(s) in the PKD1 gene and/or the PKD2 of the cell.

### Cell Cultures and Cell Populations

Also provided is a primary culture of cystogenic, single cyst-derived cells from a polycystic kidney. Optionally, the primary culture of cystogenic, single cyst-derived cells can be made using any of the methods described herein. Optionally, the primary culture of cystogenic, single cyst-derived cells is produced by (a) isolating one or more cells from a single cyst in a polycystic kidney; (b) culturing the isolated one or more cells in a culture dish or well under conditions for expanding the one or more cells to create a plurality of single cyst-derived cells; (c) transferring a first subset of the plurality of single-cyst derived cells to a three dimensional culture system configured for cyst formation; (d) determining the first subset of the plurality of single-cyst derived cells form cyst-like structures in the three dimensional culture; and (e) isolating a second subset of the plurality of single cyst-derived cells to create a primary culture of cystogenic, single cyst-derived cells from a polycystic kidney. Optionally, any primary culture described herein or made by a method described herein can be immortalized.

Further provided are populations of cells passaged from a primary culture described herein. Optionally, the population of cells is a population that has been passaged one, two, three, four, five or more times. Optionally, the population of cells is a population that has been passaged fewer than five, four, three or two times.

Also provided is a population of immortalized, cystogenic, single cyst-derived cells from a polycystic kidney. Optionally, the population is a population derived using any of the methods for producing an immortalized cystogenic, single cyst-derived cells as described herein. Also provided is a 3D culture of immortalized, cystogenic, single cyst-derived cells from a polycystic kidney comprising (a) a population of immortalized, cystogenic, single cyst-derived cells and (b) a 3-D gel. In some examples, the population of immortalized, cystogenic, single cyst-derived cells in the 3D culture is a population of immortalized, cystogenic single cyst-derived cells produced by any of the methods provided herein.

Specific immortalized, cystogenic, single cyst-derived cells from a polycystic kidney are useful for transepithelial electrical assays such as transepithelial electrical resistance and voltage assays under open-circuit conditions, fluid transport assays, and Ussing chamber-driven electrophysiology. The cells are growth on permeable filter supports for these bioassays.

### Screening Methods

Any of the primary or immortalized cell cultures described herein can be used to identify one or more agents that decrease or reduce cyst-like formation. A decrease in cyst-like formation can be a decrease in the number of cysts, a decrease in growth of an existing cyst, a decrease in the growth rate of an existing cyst, or a delay in the appearance of one or more cysts. Provided herein is a method of screening for an agent that prevents or treats polycystic kidney disease comprising (a) contacting a culture of cystogenic, single cyst-derived cells from a polycystic kidney with an agent to be screened; (b) transferring the contacted cells to a three dimensional culture system configured for cyst formation; and (c) determining the level of cyst-like formations in the three dimensional culture system of the contacted cells after treatment with the agent as compared to a control level. A reduced level of cyst-like formations indicates the agent prevents or treats polycystic kidney disease.

Further provided is a method of screening for an agent that prevents or treats polycystic kidney disease comprising (a) contacting a 3D culture system of cystogenic, single cyst-derived cells from a polycystic kidney with an agent to be screened and (h) determining the level of cyst-like formations in the three dimensional culture system of the contacted cells after treatment with the agent as compared to a control level. A reduced level of cystogenic capacity indicates the agent prevents or treats polycystic kidney disease.

Optionally, the 3D culture system of cystogenic, single cyst-derived cells from a polycystic kidney is contacted with the agent after two or more cysts have formed in the 3D culture system, for example, after one, two, three, four, five, six, seven, eight, nine, ten or more days of cell growth in the 3D culture system. Once sizable cysts have formed, the 3-D system is contacted with the agent and the level of cyst-like formations in the 3D culture system is determined. A reduced level of cystogenic capacity, for example, fewer cysts and/or smaller cysts, as compared to a control, indicates the agent treats polycystic kidney disease.

The level of cystogenic capacity can be determined 1, 2, 3, 4, 5, 6, 7, 8, 9, 10, 11, 12, 13, 14, 15, 16, 17, 18, 19, 20, 21, 22, 23, 24, 25, 26, 27, 28 days or later after contacting the 3D culture system with the agent. The reduction in cystogenic capacity does not have to be complete as this can range from a decrease in cytogenic capacity to complete ablation of one or more existing cysts. Thus, the reduction can be a 10%, 20%, 30%, 40%, 50%, 60%, 70%, 80%, 90%, 100%, or any percent reduction in between 10% and 100% as compared to native or control levels.

Treating or treatment of any disease or disorder refers to ameliorating a disease or disorder that exists in a subject. The term ameliorating refers to any therapeutically beneficial result in the treatment of a disease state, e.g., polycystic kidney disease, lessening in the severity or progression, promoting remission or durations of remission, or curing thereof. Thus, treating or treatment includes ameliorating at least one physical parameter or symptom, for example, reduction in the size of one or more cysts in the kidney of a subject. Treating or treatment includes modulating the disease or disorder. Treating or treatment includes delaying or preventing progression of polycystic kidney disease. It is understood that treatment does not necessarily refer to a cure or complete ablation of the disease, condition, or symptoms of the disease or condition.

Optionally, the 3D culture system of cystogenic, single cyst-derived cells from a polycystic kidney is contacted with the agent before or when cysts have just formed in the 3D culture system, for example, before one, two, three, four, five, six, seven, eight, nine or ten days of cell growth in the 3D culture system. Optionally, the culture system is contacted with the agent when two or more cysts have first formed. Optionally, when performing the assay in multiple yells or dishes, the culture system is contacted with the agent when an average of three cysts per culture system first form. The level of cystogenic capacity can be determined 1, 2, 3, 4, 5, 6, 7, 8, 9, 10, 11, 12, 13, 14, 15, 16, 17, 18, 19, 20, 21, 22, 23, 24, 25, 26, 27, 28 days or later, after contacting the 3D culture system with the agent. A reduced level of cystogenic capacity, for example, fewer cysts, or smaller cysts, as compared to a control, indicates the agent prevents the development of polycystic kidney disease. In some examples, the control is the cystogenic level of untreated cells derived from the same cyst

Optionally, any of the screening methods provided herein can further comprise contacting normal human kidney cells with the agent to determine the toxicity of the agent. For example, when transferred or embedded in a 3D culture system, normal human kidney cells generate tubule or duct-like networks. Sometimes, the normal human kidney cells form small cysts and spheroids. High content imaging is performed to visualize the cysts, spheroids, tubules and other structures daily and over time. In methods comprising screening for toxicity, one or more agents can be contacted with a 3D culture system comprising normal human kidney cells and the level of tubulogenesis can be assessed, prior to screening the agent for cystogenic activity. Optionally, if the agent is toxic, the agent is not screened for cystogenic activity. A 50% reduction in tubulogenesis to elimination of tubule structures is considered cytotoxic in this assay. These assays thus identify cytotoxic agents and chemicals or small molecules that are toxic to renal cells and tissues.

Optionally, any of the screening methods provided herein can further comprise determining the level of inflammatory factors secreted by the cells after being contacted with the agent. A decrease in the production or secretion of one or more inflammatory factors indicates that the agent inhibits inflammation associated with polycystic kidney disease. Monocyte chemoattractant protein 1 (MCP-1), interleukins 8 and 6 (IL-8 and IL-6), and tumor necrosis factor alpha (TNFalpha) are commonly hypersecreted by cystogenic, single cyst-derived cells from a polycystic kidney.

In any of the screening methods provided herein, the agent can be, but is not limited to, a chemical, a small or large molecule (organic or inorganic), a drug, a protein, a peptide, a cDNA, an antibody, a morpholine, a triple helix molecule, an siRNA, a shRNA, an miRNA, an antisense RNA or a ribozyme to name a few. Optionally, in any of the screening methods provided herein, the culture of cystogenic, single cyst-derived cells from a polycystic kidney comprises a primary culture of cells isolated from a single cyst or immortalized cells. Optionally, the culture comprises immortalized, cystogenic, single cyst-derived cells from a polycystic kidney Optionally, in any of the screening methods provided herein, the method can further comprise correlating the agent that prevents or treats polycystic kidney disease with a specific genetic profile of the cystogenic, single cyst-derived cells. Optionally, in any of the screening methods provided herein, the control level is the cystogenic level of untreated cells derived from the same cyst.

Disclosed are materials, compositions, and ingredients that can be used for, can be used in conjunction with or can be used in preparation for the disclosed embodiments. These and other materials are disclosed herein, and it is understood that when combinations, subsets, interactions, groups, etc. of these materials are disclosed that while specific reference of each various individual and collective combinations and permutations of these compositions may not be explicitly disclosed, each is specifically contemplated and described herein. For example, if a method is disclosed and discussed, and a number of modifications that can be made to a number of molecules included in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties. The following description provides further non-limiting examples of the disclosed compositions and methods.

### EXAMPLES

### Media

500 ml bottles of Ca^{2+/}Mg²⁺⁻free phosphate buffered saline (PBS) (or Hanks' Balanced Salt Solution (HBSS)), with 5× antibiotics ((25 mls of 100× Pen/Strep stock solution (Corning Fisher Scientific (Hampton, N.H.), 10 mls of Fungizone (Amphotericin B) stock solution (Corning Fisher Scientific), and 1 ml of Gentamicin stock solution (Corning Fisher Scientific) were prepared.

At least two liters of Ca^{2+/}Mg²⁺⁻ free DMEM/F-12 Ham's media (14.8 grams of powder media were mixed with 1 liter of distilled water and filter sterilized with a 1 liter filter bottle system) (Sigma-Aldrich (St. Louis, Mo.)) with 5× antibiotics (as above), with 5 mls of added L-Glutamine 100× stock solution were prepared.

500 ml bottles of Advanced MEM (Gibco-BRL (Hampton, N.H.), Invitrogen (Carlsbad, Calif.)), with 5% FBS, 5× antibiotics (as above) and 5 mls of added L-Glutamine 100×, stock solution were prepared. This media was used for washing the tissues after each digestion.

At least one liter of Ca^{2+/}Mg²⁺⁻ free DMEM/F-12 Ham's media with 5× antibiotics (as above), L-Glutamine (as above) and 0.5 nag/ml Collagenase P (Roche, Basel, Switzerland) was prepared to create Digestion Media for 4° C. and 37° C. digestions.

Several liters of DBM RenalCyte media supplemented with 5× antibiotics were prepared.

### Processing the Whole Kidney Specimen

The human kidney tissue specimen was removed from triple-bagged containers on ice. Excess liquid was aspirated. Ca^{2+/}Mg²⁺⁻ free PBS was poured over the tissue to wash/moisten the tissue. Any excess adipose tissue was dissected away from the tissue. This was minimal to quite substantial and was donor dependent.

### Dissection of ADPKD (or ARPKD) Kidneys

End-stage human ADPKD kidney tissue fragments or whole kidneys were quite large and fully compromised with fluid-filled cysts. The large cysts that can be individually dissected were readily apparent. The initial dissection emphasized isolating as many single cysts as possible, with each cyst going into their own separate culture vessel, immediately after dissection. Each cyst was kept separate, in primary culture, from that point on. The steps below describe the dissection of a single isolated cyst.

Large cysts were individually dissected from the tissue. Often, course scissors were needed to cut through tough connective tissue surrounding the largest cysts. Cysts often held clear fluid, blood or cloudy fluid. Collection of clear fluid-filled cysts were prioritized but blood colored fluid-filled cysts were also collected. Cloudy fluid-filled cysts were generally avoided.

Each single cyst was placed into its own Petri dish and washed again with Ca^{2+/}Mg²⁺⁻⁻free PBS with 5× antibiotics. Extraneous tissues were then dissected from the surface of each cyst, and any fluid inside the cysts was dispelled. Upon removal of fluid, the cyst collapsed. The cyst was washed again with Ca^{2+/}Mg²⁺⁻+-free media including 5× antibiotics and partially Minced. Then, the cyst wall tissue fragment was placed into cold Digestion Media, typically, in a 50 ml conical tube, and the minced tissue from each cyst was placed into 20 ml of Digestion Media. The cyst tissue was incubated in Digestion Media, in the 50 ml conical tube, overnight at 4° C. This procedure was repeated for as many individual single cysts as possible with Ca^{2+/}Mg²⁺⁻free PBS poured over the tissue many times during dissection. Once all desired single cysts were individually dissected and placed into separate Petri dishes and conical tubes, multicystic, microcystic tissue sections were dissected out of the donor kidney tissue.

Sections of multicystic/microcystic kidney tissue were dissected out. Typically, these were clusters of very small cysts within the tissue. A section of such tissue was placed into a Petri dish. The section was washed with Ca^{2+/}Mg²⁺⁻-free PBS and any excess adipose tissue or connective tissue was dissected away. The tissue was washed again with Ca^{2+/}Mg²⁺⁻-free media and minced with scissors. The minced tissue was then placed into a 50 ml conical tube filled with 20 mls of Digestion Media and incubated overnight at 4° C.

### Dissection of Normal and Diabetic Human Kidneys

Normal and diabetic human kidney specimens were closer to normal in size, although diabetic human kidneys can display hypertrophy and become larger. After washing, as described above, the kidney was cut longwise and in half down the long axis of the kidney, to obtain two halves of the kidney with the inner regions of the kidney exposed. The inner medulla, the white matter near the point where the collecting ducts coalesce, was dissected away from the rest of the kidney tissue and processed as inner medulla primary cultures. The outer medulla, (the pink or red portions where the pyramidal calyxes of the medulla reside) was dissected separately and processed into its own primary culture. The majority of the kidney tissue (that which remained after dissecting away medullary regions) was the cortex of the kidney. The cortex was dissected in sections or slices and processed into primary cultures. In some procedures, there were about two to four Petri dishes of inner medulla tissue, three to six Petri dishes of outer medulla tissue, and as many as 16 Petri dishes of cortex to process. Each section of tissue was placed into a Petri dish and washed with Ca^{2+/}Mg²⁺⁻free PBS. Any excess adipose tissue or connective tissue was dissected away prior to washing the tissue again with Ca^{2+/}Mg²⁺⁻free media. Scissors were used to mince the tissue. The minced tissue was placed into a 50 ml conical tube filled with 20 mls of Digestion Media and incubated overnight at 4° C.

Primary Cell Collections from Tissue Digestions

For all types of tissue digested from diseased or normal human kidneys, the following step-wise process was used for collecting cell pellets from the digestions over time.

Tubes were removed from the refrigerator and 5 mls of straight fetal bovine serum (FBS) were added to each tube to stop collagenase P activity. Gentle agitation was used to mix in the FBS. Then, each 50 ml conical tube with digesting tissue was inverted 20-25 times to agitate before allowing the tissue to settle to the bottom of the tube. The supernatant was pipetted off and 20 mls of Advanced MEM (plus 5% FBS plus 5× antibiotics) were added to each conical tube with tissue pieces. Each tube was inverted again about 20-25 times to agitate and supernatant was again removed and collected with the first supernatant. The supernatants were centrifuged for 5 minutes at 2400 RPM. Large cell pellets, with a red coloring due to the presence of RBCs, appeared at the bottom of the tube. Most of the supernatant was removed, leaving behind a bit of liquid to break apart the cell pellet with vortexing. One ml of DBM RenalCyte media with 5× antibiotics was added to resuspend the cells with a bit of fresh media to rest the cells before seeding into T175 flasks. The entire cell pellet was seeded into a T175 flask. This was done for each cell pellet for each primary cell culture being generated.

A second digestion was performed at 37° C., in a cell culture incubator, for one hour. The steps of the first digestion were repeated. A third digestion was performed at 37 CC in a cell culture incubator for another hour. Twenty mls of Ca^{2+/}Mg²⁺⁻free DMEM/F12 (Digestion Media) with 0.5 mg/ml Collagenase P kept at room temperature were added to the tubes still containing the tissue to be digested. Then, the tubes were placed in the cell culture incubator for a final hour and the steps of the first digestion were repeated.

At this point, there were 3 T175 flasks for each primary culture established. In some cases the digested tissue was pipetted up and down to break it apart and smaller pieces that could be pipetted into a 4th T175 flask were seeded to establish a Cyst 1 Explant culture.

### Passage 0 (p0) Primary Cell Culture

The T175 flasks were placed in incubators and left undisturbed for four days to allow all viable cells to attach well to the substrate. Small tissue fragments also attached and cells grew out. After the 4-day cell attachment phase, the flasks are washed with Ca^{2+/}Mg²⁺⁻containing HBSS with 5× antibiotics to clean the primary cultures of any and all unattached debris. As many as three washes were required to clean the primary cultures. At this point, attached and viable cells were visible. Images were taken and percent confluence was assessed.

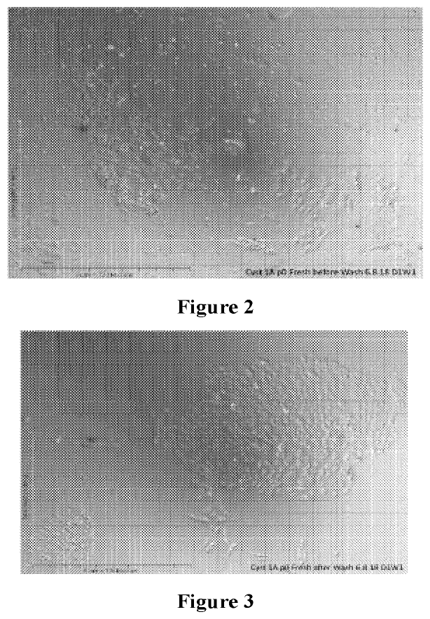

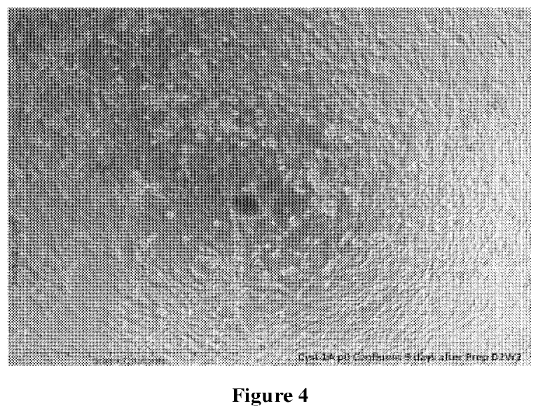

After the washes, fresh DBM RenalCyte media was added (20-24 mls) to re-feed the cultures. Cells reached confluence as fast as 8-9 days after seeding, but in some cases took 2-3 weeks. Once a T175 flask was confluent, the flasks were washed once in Ca^{2+/}Mg²⁺⁻-free PBS with 5× antibiotics and then incubated in 18 mls of the same for at least 1 hour in a 37° C. incubator. After the Ca^{2+/}Mg²⁺⁻free PBS incubation step, the flasks were hit multiple times to loosen the attached cells. Loosened cells were collected in a 50 nil conical tube and 10 rats of DBM RenalCyte media were added to keep the cells fed. Then, 12 mls of trypsin-EDTA solution were added. The flasks were then incubated until all of the cells attached. After the trypsin-EDTA incubation step, the flasks were hit multiple times and loosened cells were collected in the same 50 ml conical tube with the PBS-loosened cells and media. Afterwards, the cells were pelleted by centrifugation for 5 minutes at 2400 RPM. The supernatant was poured off, but a little liquid was left to break apart the cell pellet with vortexing. One ml of DBM RenalCyte media was added. A 5 μl aliquot of the cell suspension was mixed with 5 μl of Trypan Blue and counted in the Invitrogen Countess II machine. At this point, an aliquot of the cells was seeded into a new flask for passage 1 or the cells were used in an assay. A 10 μl aliquot of the cells was pipetted into a separate small tube and mixed with 300 μl of cold DBM 3D Biogel solution to form a 3D Biogel. The cells were then tested for cystogenic or tubulogenic phenotype. FIG. 2 shows a primary huADPKD single cyst culture after plating and before any washes. FIG. 3 shows a primary huADPKD single cyst culture after washings. FIG. 4 shows a primary huADPKD single cyst culture well that was confluent after 9 days.

### Cryopreservation of Primary Cell Cultures

Upon cell counting, there were often at least 10 million cells derived from a very-confluent T175 flask, although this number varied. With the cell pellet already resuspended in 1 ml of DBM RenalCyte media, cold DBM Cryopreservation Media was added, using 1.5 mls for every cryovial. Generally there were greater than one million cells per cryovial, but this number can vary. DBM Cryopreservation Media, Lifeline Cell Technology's FrostaLife (Oceanside, Calif.) or Sigma-Aldrich's CryoSOfree media can be used.

### Extending the Lifetime of Primary Cultures

The lifetime of the primary cultures grown on standard tissue culture plasticware is about four passages. However, by growing the primary huADPKD cystic kidney cells on permeable filter supports, the lifetime of the same primary cultures is extended to at least eight passages. The practice of growing and maintaining primary huADPKD cystic cells and primary nominal human kidney cells on permeable filter supports also helps maintain the ability of the cells to form cysts or tubules, respectively, in a 3D tissue culture system, for example a 3D Biogel. In some cases, the cells were grown on Corning Costar 3450-6 plates wherein each well contains a large 24 mm clear polyester filter support, which allows visualization of the cells during expansion. The cells were optionally grown on a Corning 2419 10 cm dish permeable filter support made from polycarbonate.

### Results of Processing and Cryopreservation Methods

Two 10-lb diseased human ADPKD) (huADPKD) kidneys from a donor were received and processed into 50 single cyst-derived primary cultures and 8 multicystic microcystic tissue-derived primary cultures. In total, approximately 1,500 cryovials of 1 million cells per cryovials were expanded and cryopreserved as passage 0 and passage 1 cells. With standard re-derivation on permeable filter supports, these primary cultures can be well maintained as early passage cells.

The second digestion and second wash huADPKD cell pellets yielded better primary cell yield in seeding large T175 flasks. The first digestion and first wash yielded cells as well; however, the cultures were slower to reach confluence and had higher fibroblast content.

There were 4 groups or waves of cells that were fast to slow in terms of being cryopreserved and seeded into 3D biogels to assess cystogenic phenotype. In general, the first two ‘waves’ of primary cultures were cystogenic and displayed the proper mixture of cell types. The slower two waves' had higher fibroblast content and may need to be subjected to differential trypsinization to lower the fibroblast number; but, many of these cultures still made cysts. Those cultures that are too fibroblastic can be used to assess lead therapeutic assets for aggressive hyperproliferative fibroblast anti-proliferation assays. Overall, after assessing cystogenecity, there were many primary cultures (the majority) that were cystogenic. Those deemed ‘OK with higher than desirable fibroblast content’ were subjected to differential trypsinzation and improved to cystogenesis assays. Those with an unacceptably high fibroblast percentage can be used for anti-proliferation assays. Some of the cultures were used to assay for secreted chemokine inhibition and for anti-proliferative effects against aggressive hyperproliferative fibroblasts after administration of a test compound.

### Immortalization of Primary Cell Cultures

High-titer aliquots (10⁹ pfu) of Lenti-SV40T, Lenti-hTERT-Neo and Lenti-CDK4 from Applied Biological Materials (ABM) (Richmond, BC, Canada) and Lenti-CDNK2 (Sigma-Aldrich) were diluted into transfection media to 10⁷ pfu for cell challenge. In some cases, Lenti-hTERT and Lenti-CDK4 or Lenti-CDNK2 were combined as a newer immortalization method. huADPKD primary cultures were selected for immortalization based on a compelling genotype, an acceptable cell proliferation rate, and an ability to form cysts in 3D Matrigel culture. Mixed immortalized cultures were established by both methods from primary cultures where the lifetime of the cells were doubled from 5 passages (primary) to 10 passages (mixed immortal). The SV40T method was successful in generating immortal cell cultures that formed electrically tight polarized cell monolayers for transepithelial resistance (TEER) measurements and likely Ussing chamber-based electrophysiological recording of ion transport. These immortal human ADPKD cell monolayers may also be useful in fluid transport assays. Transepithelial/transendothelial electrical resistance (TEER) data for a new immortalized human ADPKD cell line are shown in Table 1 below. The increase in resistance with amiloride treatment shows that the epithelial sodium channel (ENaC) is expressed in the cell monolayers.

TABLE 1
TEER Data for Immortal Human ADPKD Cell Line

| 6-10326:p5 | | Monolayer TEER Dev | | | | 6-10326:p4 | | Monolayer TEER Dev | | | |
|---|---|---|---|---|---|---|---|---|---|---|---|
| Pre-Amiloride | Post-Amiloride | delta | Day 3 | 185 | 183 | Pre-Amiloride | Post-Amiloride | delta | Day 3 | 199 | 187 |
| 842 | 978 | 136 | Day 5 | 240 | 235 | 952 | 1235 | 283 | Day 5 | 345 | 352 |
| 731 | 805 | 74 | Day 7 | 347 | 318 | 991 | 1189 | 198 | Day 7 | 675 | 682 |
| 731 | 818 | 87 | Day 12 | 735 | 618 | 1024 | 1345 | 321 | Day 12 | 789 | 795 |
| 724 | 810 | 86 | Day 14 | 772 | 775 | 1086 | 1401 | 315 | Day 14 | 977 | 970 |
| 724 | 794 | 70 | | | | 1011 | 1359 | 348 | | | |
| 772 | 855 | 83 | | | | 987 | 1276 | 259 | | | |
| 774 | 855 | 121 | | | | 965 | 1253 | 288 | | | |
| 740 | 832 | 92 | | | | 897 | 1109 | 212 | | | |
| 548 | 716 | 78 | | | | 1123 | 1453 | 330 | | | |
| 690 | 765 | 75 | | | | 1054 | 1386 | 332 | | | |
| 542 | 725 | 78 | | | | 977 | 1276 | 299 | | | |
| 775 | 835 | 60 | | | | 976 | 1288 | 312 | | | |
| 733.2 | 819.0 | 86.7 | | | | 1003.6 | 1297.5 | 291.4 | | | |
| 55.2 | 71.4 | 21.5 | | | | 61.4 | 96.8 | 47.1 | | | |

All values are transepithelial resistance in Ohms, shown as mean ± S.D. for cultures pre- and post-amiloride.

### Genotyping

With the Human Genetics Cores in the Mayo Clinic PKD Center and at the University of Arizona, flash frozen cell pellets of our primary human ADPKD cell cultures (mainly, the single cyst-derived cultures) were sent to the Cores, who isolated the genomic DNA and either used the Sanger method of DNA sequencing or next generation sequencing of the PKD11, PKD2, and PKHD1 genes or, in more rare cases, whole genome sequencing (WGS) if it was unclear as to whether the kidney was a bona fide polycystic kidney. Table 2 shows the results of genotyping studies.

TABLE 2
DBM Human ADPKD Primary Cultures by Donor with Genetic Mutation Analysis

| Donor # | Tissue(s) | Primary Cultures | Germline Mutation/Somatic Mutation |
|---|---|---|---|
| Donor 1 | Partial kidney | 5 SC; 2 MCT | PKD1 P2736R, L2763V, |
| | | | M2764T (No second |
| | | | somatic mutations) |
| Donor 2 | Partial kidney | 7 SC; 5 MCT | PKD1 E685 frameshift/ |
| | | | truncation (Second somatic |
| | | | mutation only in one |
| | | | primary culture, SC#3, |
| | | | was PKD1 P3448 frameshift/ |
| | | | truncation only) |
| Donor 3 | Both kidneys | 18 SC; 8 MCT | PKD2 P872X nonsense |
| | | | truncation (Frequent second |
| | | | somatic mutations: SC#1 |
| | | | A62 frameshift/truncation; |
| | | | SC#2 L237 frameshift/truncation; |
| | | | SC#4 L715 frameshift/truncation; |
| | | | SC#7 A62 frameshift/truncation; |
| | | | SC#12 Q160 frameshift/truncation; |
| | | | SC#14 K718 frameshift/truncation; |
| | | | SC#17 Q542 frameshift/truncation; |
| | | | SC#18 Y684 frameshift/truncation) |
| Donor 4 | Both kidneys | 14 SC; 9 MCT | PKD1 R2757 frameshift/truncation |
| | | | (Second somatic mutations only |
| | | | in two cases: SC#7 N116 |
| | | | frameshift/truncation; |
| | | | SC#11 G617 frameshift |
| | | | (silent splice) |
| Donor 5 | Partial kidney | 8 SC; 0 MCT | PKD1 G3690E missense |
| | | | mutation (No second |
| | | | somatic mutations) |
| Donor 6 | Both kidneys* | 24 MCT | PKD1 and PKHD1 dual |
| | | | mutations (Second |
| | | | somatic mutations |
| | | | not evaluated^{#}) |
| Donor 7 | Both kidneys | 50 SC; 8 MCT | PKD1 1202 − 9G > A |
| | | | Common Splice Mutation |
| | | | (No second somatic mutations) |
| Donor 8 | Single kidney** | 4 SC, 12 NHK^{$} | In progress, WGS*** |
| Donor 9 | Both kidneys | 16 SC, 27 MCT | PKD1, verification in |
| | | | progress (Second hits not |
| | | | yet evaluated) |
| Donor 10 | Both kidneys | 20 SC, 20 MCT | PKD1 (c. 566C > G p.Ser189) |
| | | | nonsense mutation) |
| Donor 11 | Both kidneys | 14 SC; 49 MCT; 10 NHK^{$} | In progress |
| | | 2B cells | |
| Donor 12 | One kidney | 16 SC | In progress |

*Both kidneys were obtained from Donor 6 who was a younger donor and where there were 100s of microcysts in each kidney but no larger, more dominant cysts that had broken through the renal capsules. It was not a typical looking ADPKD kidney and it bears both PKD1 and PKHD1 mutations, making it a mixed PKD genotype which does occur, but rarely. However, no individual cysts were large enough to dissect out separately into distinct primary cultures.
**Donor 8 was a young pediatric donor advertised as an ARPKD patient; however, there was only one very large cyst in the kidney, a few smaller cysts around it, and the rest of the tissue was non-cystic. All cultures from this donor kidney are exceedingly hyperproliferative but disease etiology is confusing because single cysts have been observed within ‘normal’ human kidneys isolated for other initiatives.
^{$}Donors 8 and 11 both had regions or ‘pockets’ within the diseased kidney that were non-cystic. This is difficult to identify/dissect from later stage or end stage ADPKD tissue specimens.

As shown in Table 2, twelve donors have been processed. In Table 2, SC=single cyst-derived primary culture; MCT=microcystic multicystic tissue-derived primary culture; and NHK=primacy cultures derived from non-cystic tissue identified within an ADPKD tissue specimen.

Single cyst-derived cultures were genotyped for germline mutations as well as any secondary somatic mutations. The results for seven donors show the following: no somatic mutations were found in 3 donors. 1-2 instances of somatic mutations were found in 2 donors, and one single PKD2 mutant-bearing donor had frequent examples of secondary somatic hits which were different from individual cyst culture to cyst culture. The mutations are donor dependent. In some instances, ADPKD patients may have efficient enough gene editing to prevent somatic mutations while others may not.

### Cyst Prevention Cytogenesis Bioassay

Primary cells capable of 3D cystogenesis were seeded into a 3D biogel that is liquified before the gel sets. The gel set undisturbed within a humidified 5% CO₂ incubator for 18 hours. An equal volume of DBM RenalCyte media was then overlaid in each well of a 48-well plate. “Add” feeds were performed until the well was full, and then half of the volume of media was removed and new media added at the same volume thereafter. Seven to ten days were required for cysts to appear (based on visual inspection with light microscopy). In the cyst prevention assay, cysts were challenged as soon as they started to appear (small in dimension and few in number per well) with a test compound. The timing of addition of the test compound, frequency of addition of the test compound and concentration of the test compound varied. The effects on the cysts were followed by light-based microscopic imaging over time. In addition to visual inspection by light-based microscopic imaging occurring throughout the study, the number of cysts were counted per well at the end of the study and light-based endpoints were examined, such as 3D CellTiter-GLO® from Promega (Madison, Wis.) (to quantify viable cells), secretion (to quantify cell membrane integrity), and/or Caspase-GLO® (Promega) (to measure induction of apoptosis) in multiplex fashion. A modified version of this assay images after seeding the biogel each day for complete prevention of cyst formation versus counterpart controls over a 14 to 21-day period. Multiple replicates were performed, and cells from multiple donors were tested.

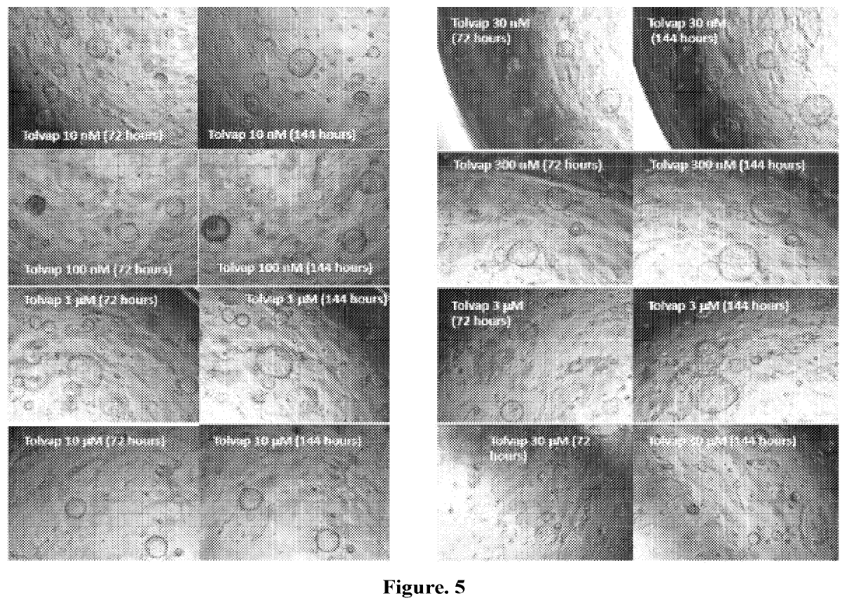

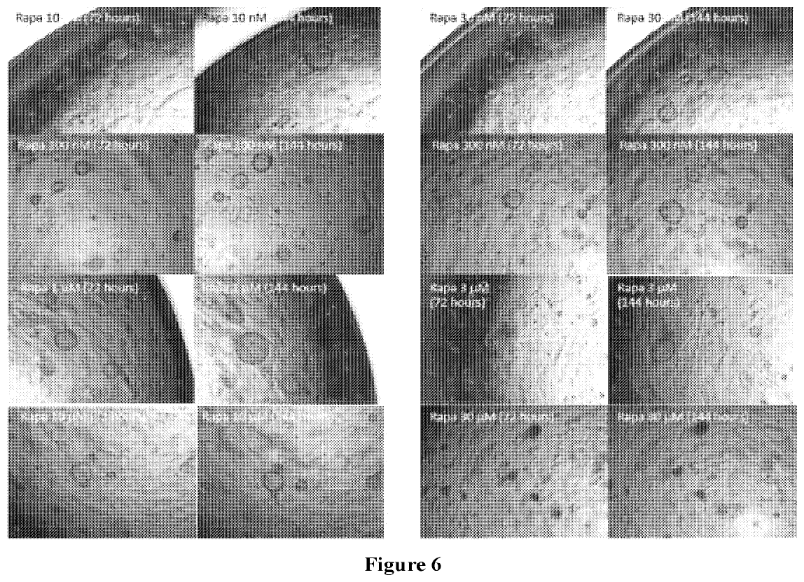

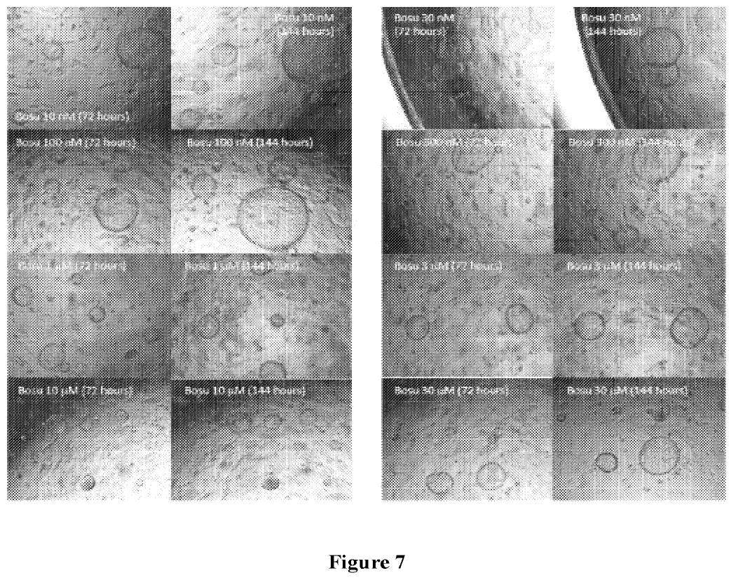

As shown in FIG. 5, at the midway point and at the end of the cyst prevention assay, the industry standard drug, tolvaptan, had little effect on cyst appearance and expansion. This vasopressin receptor antagonist may have slowed cyst expansion at 10 and 30 micromolar, however, the solubility limit for this drug was near 30 micromolar. There was no overt cell cytotoxicity with tolvaptan. As shown in FIG. 6, at the midway point and at the end of the cyst prevention assay, the industry standard drug, rapamycin, had little effect on cyst appearance and expansion. This mTORinhibitor slowed cyst expansion at 10 micromolar; however, the solubility limit for this drug is near 30 micromolar. There was overt cell cytotoxicity with rapamycin at 30 micromolar. As shown in FIG. 7, at the midway point and at the end of the cyst prevention assay, the industry standard drug, bosutinib, had no effect on cyst appearance and expansion. This MAP kinase inhibitor was a disappointing industry standard control. There was no overt cell cytotoxicity with bosutinib.

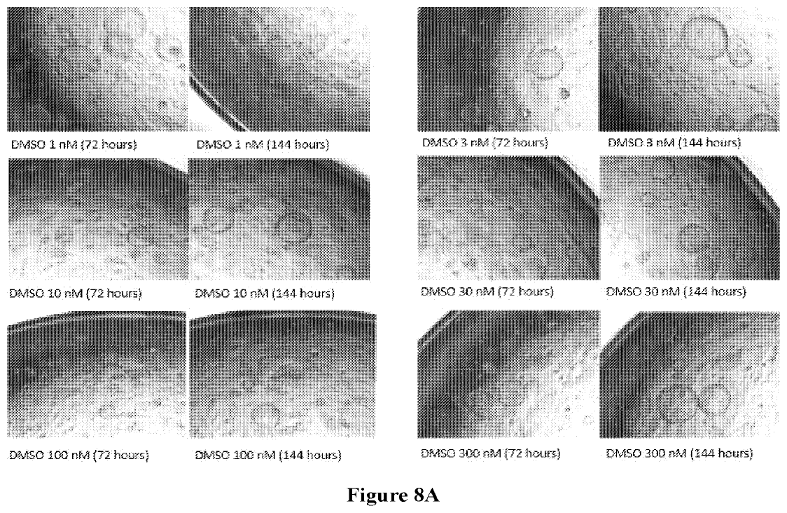

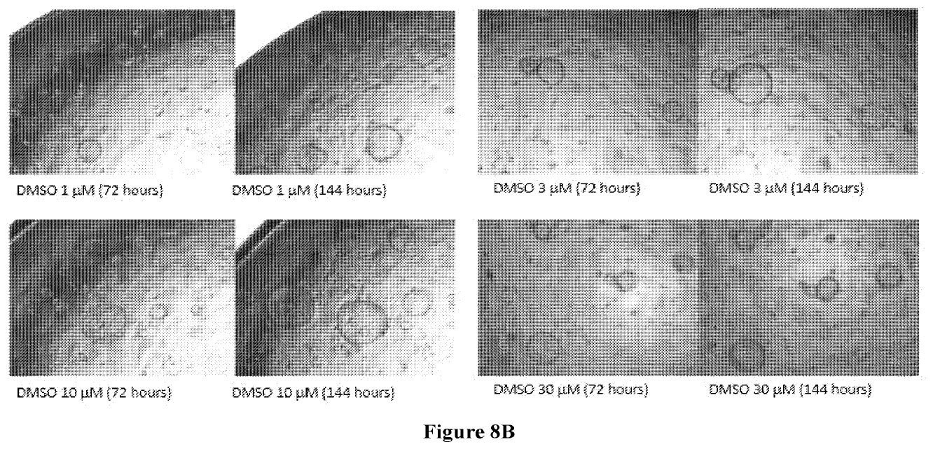

DMSO vehicle controls at concentrations of 1-30 μM were performed. Results are shown in FIG. 8. At the highest concentration of DSMO (30 μM), a modest attenuation of cyst expansion occurred between 72 and 144 hours. However, at all lower concentrations tested (1, 3, and 10 μM), the DMSO vehicle controls showed no effect in paired images of 72 hours and 144 hours.

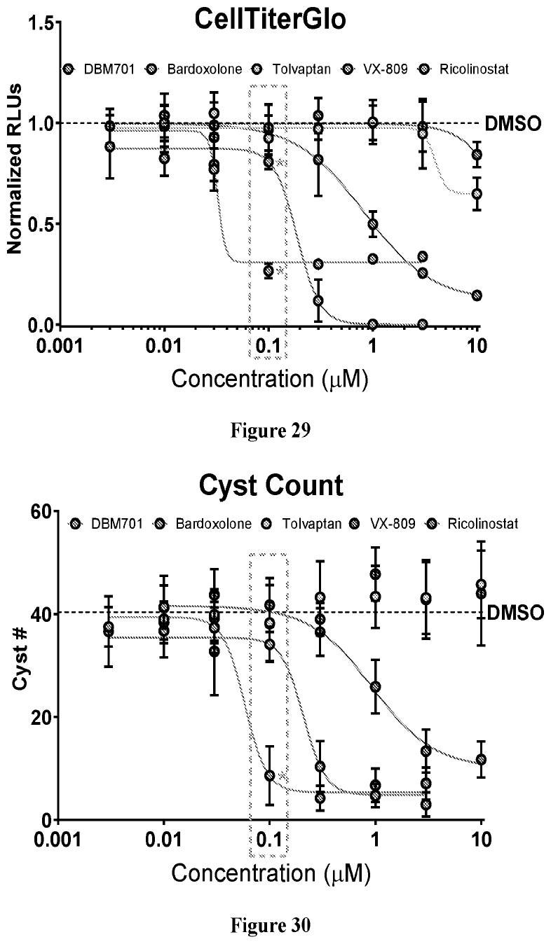

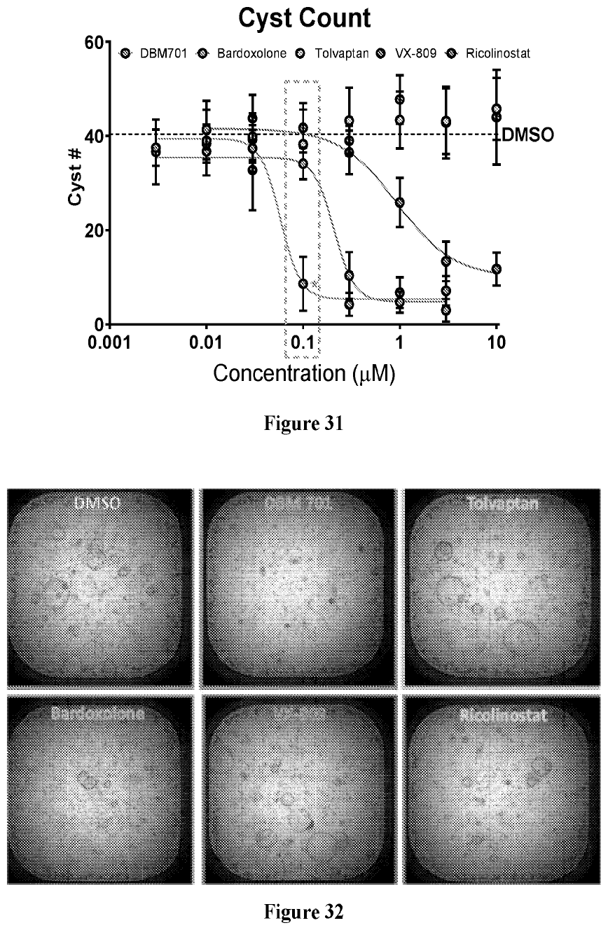

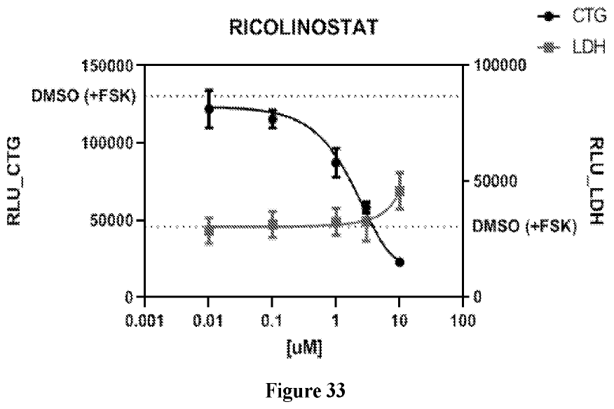

In another exemplary cyst prevention assay, 3D CellTiter-GLO® was used to quantify viable ADPKD cells in a biogel, after thirteen days of exposure to concentration-response curves of selected compounds in a compound screening assay. The results are shown in FIG. 29. In addition to cell viability, the number of cysts from ADPKD cells grown in a 3D prevention assay format was also determined (FIG. 30). Cyst size was also measured, as shown in FIG. 31. Representative images of ADPKD cells grown in the 3-D cyst prevention assay for 14 days after exposure to concentration response curves of selected compounds are shown in FIG. 32, Images are shown from a concentration of 0.1 μM, where no cytotoxicity was observed for any of the candidate compounds. FIG. 33 shows multiplexed detection of cell viability (via 3D CellTiter-GLO® (the number of viable cells per Biogel)) and LDH secretion or leakage as a measure of overt cytotoxicity. LDH is released from the cells when there is a loss of cell membrane integrity, indicating overt cytotoxicity that cannot be ascertained directly by just the use of CellTiterGLO alone. FIGS. 29-33 show data obtained using a Cytation 5 high-content imager with a wide-angle lens to image the entire well at one time. Additional assays can be used, as described herein, in multiplexed detection assays. For example, detection of secreted inflammatory mediators or autocrine/paracrine factors (e.g., MCP-1, IL-6, extracellular ATP, etc.) can be performed in combination with a cell viability assay. It is possible to use an AlphaLISA immunoassay (Perkin Elmer (Waltham, Mass.)) to detect the secretion of a select inflammatory mediators, such as MCP-1, IL-6 and TNFalpha, in order to measure the PKD ‘inflammasome’ which is a hallmark of the human PKD disease that is not not recapitulated well in rodent models of PKD,

### Cyst Attack Cytogenesis Bioassay

Primary cells capable of 3D cystogenesis were seeded as described above for the cyst prevention cytogenesis bioassay. In the cyst attack assay, cysts were allowed to develop to 12-24 in number and allowed to expand to a significant size. Specific cysts or cyst clusters were marked in each well. A test compound was then added to attack the already formed cysts to determine effects on the cysts. Cysts were followed by light-based microscopic imaging over time. The timing of addition of the compound, frequency of addition of the compound and concentration of the compound varied. In addition to visual inspection by light-based microscopic imaging occurring throughout the study and at study end, and the number of cysts counted per well at study end, light-based endpoints were examined such as 3D CellTiterGLO (to quantify viable cells), LDH secretion (to quantify cell membrane integrity), and CaspaseGLO (to measure induction of apoptosis) in multiplex fashion. Multiple replicates were performed, and cells from multiple donors were tested.

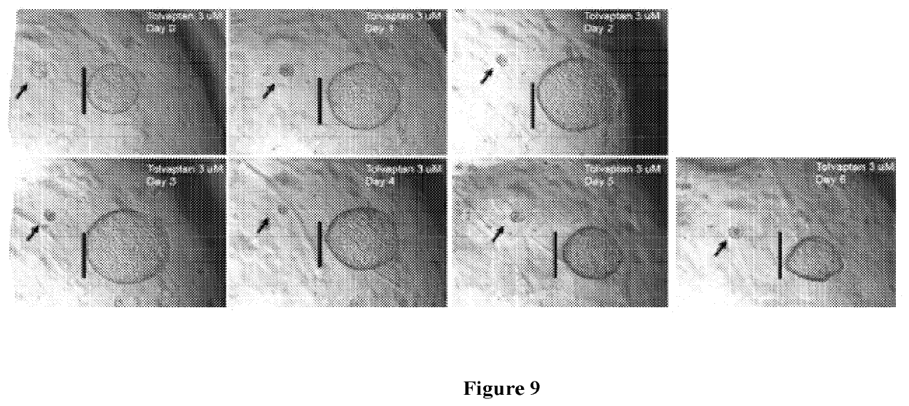

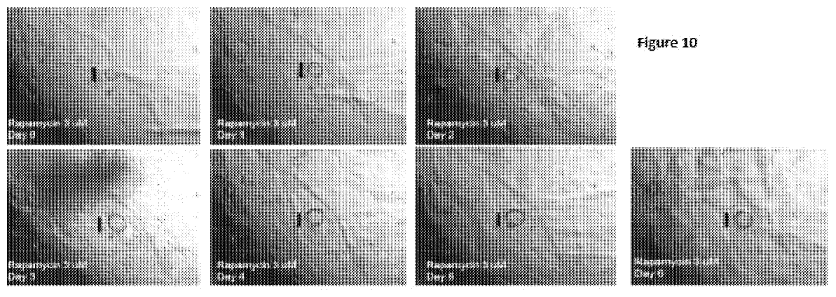

A prominent cyst was imaged before treatment and daily for 6 days after treatment began. This larger cyst increased by 2.5 to 3.0 fold in the presence of the industry standard drug, tolvaptan, a vasopressin receptor antagonist. As shown in FIG. 9, in the presence of 3 μM tolvaptan, a small cyst and a large cyst were imaged together before beginning of treatment and daily for 6 days after treatment began. The smaller cyst was quickly reduced in size (see arrow). The large cyst doubled in size but then decreased in size to a slightly reduced size at the end of a week-long treatment and lost some of its spherical shape. As shown in FIG. 10, a small cyst was imaged before beginning treatment with rapamycin (3 μM) and then daily for 6 days after treatment began. This cyst increased in size modestly and remained static for the remaining days. Rapamycin also eliminated other cysts and spheroids in other images but was not overtly toxic at this concentration. As shown in FIG. 11, a small cyst was imaged before beginning treatment with 3 μM bosutinib and then daily for 6 days after treatment began. This cyst increased in size by 2 fold in 1-2 days but was attenuated or ‘locked’ in size for days 3-6 where no further enlargement was observed.

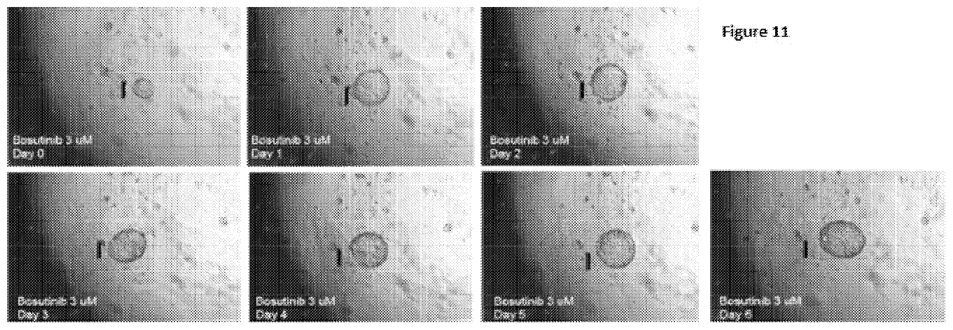

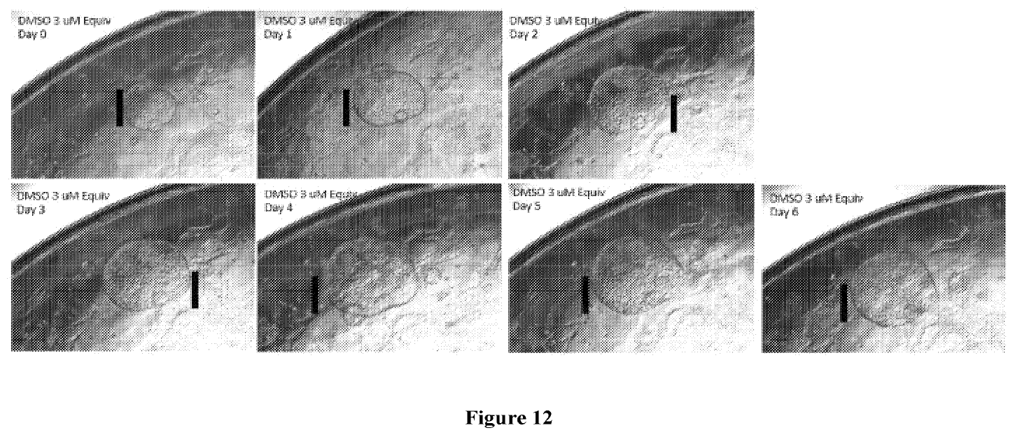

As shown in FIG. 12, using a DMSO 3 μM equivalent, a single large cyst was imaged before beginning the vehicle control and for 6 days after treatment began. That larger cyst doubled in size and additional smaller satellite cysts became more prominent or appeared in the field.

### High-Throughput Cystogenesis Assays

A BioTek Cytation 5 (Biotek instruments, Winooski, Vt.) high content imaging and light-based high-throughput screening imaging system was used to image the huADPKD cells growing in 3D Biogels in 96-well and 384-well microliter plates. The Cytation 5 is coupled to a BioTek BioSpa 8 (a humidified CO₂ incubator), such that trays can be extracted one at a time for analysis in the reader and then returned to the BioSpa. Cytogenesis was observed from key primary and immortalized huADPKD cell cultures in 96-well full area well plates, 96-well half area well plates and in 384-well plates.

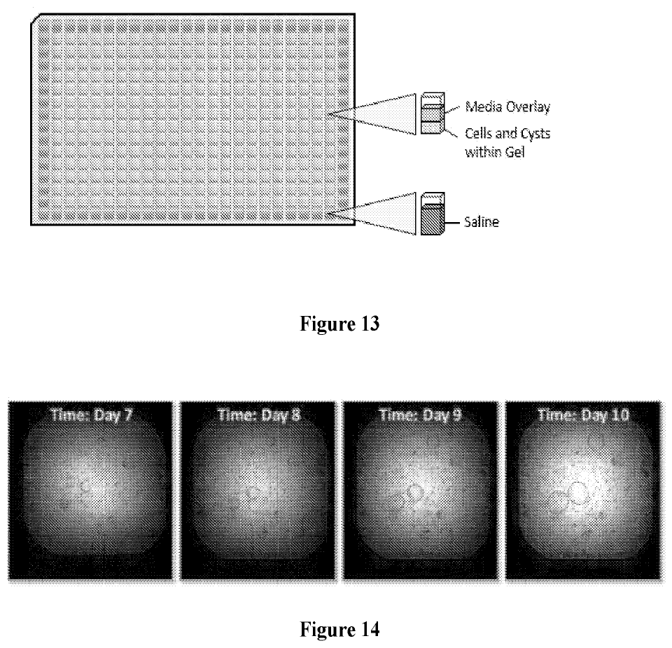

FIG. 13 shows a schematic design for a 384-well plate a cystogenesis assay. Notably, Specialty DBM RenalCyte media was sufficient to induce cystogenesis without additives (forskolin or other cAMP stimuli). While no drivers are required, they can be used if desired. Edge wells are filled with saline to mitigate edge effects

Whole well automated high-content imaging was performed of ADPKD primary cells seeded in 384-well plates using the BioTek Cytation 5 system. Multiple well were captured per treatment. Uniform images were fed into deep learning algorithms for analysis. Stimuli like cAMP agonists and growth factors were not required, as the DBM RenalCyte media was sufficient to drive cystogenesis, as shown in FIG. 14.

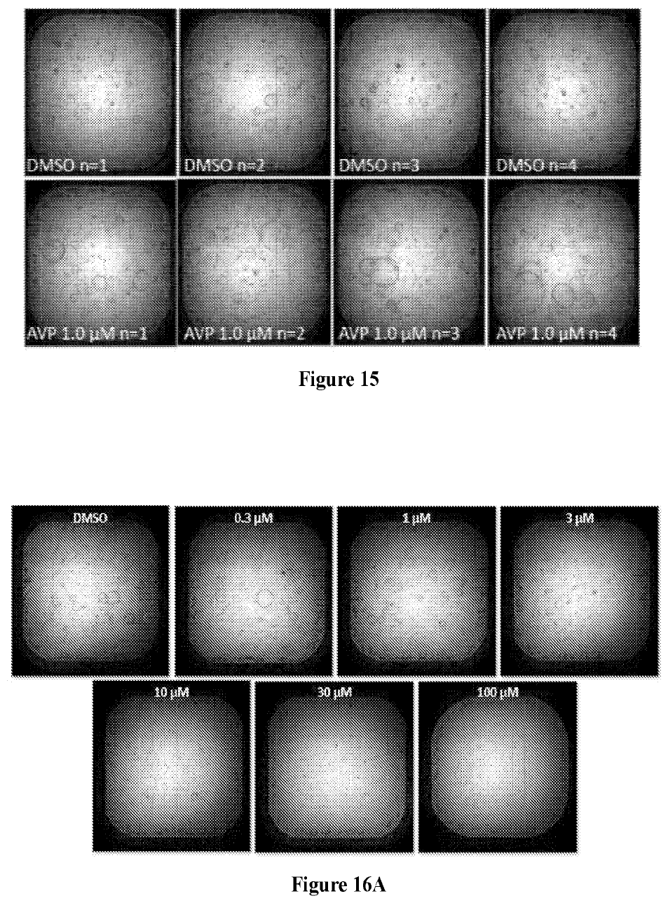

ADPKD cells were cultured for 14 days in the absence or presence of Arginine Vasopressin (AVP), As shown in FIG. 15, the cysts are more larger on average in cultures in the presence of this physiological cyclic AMP stimulus.

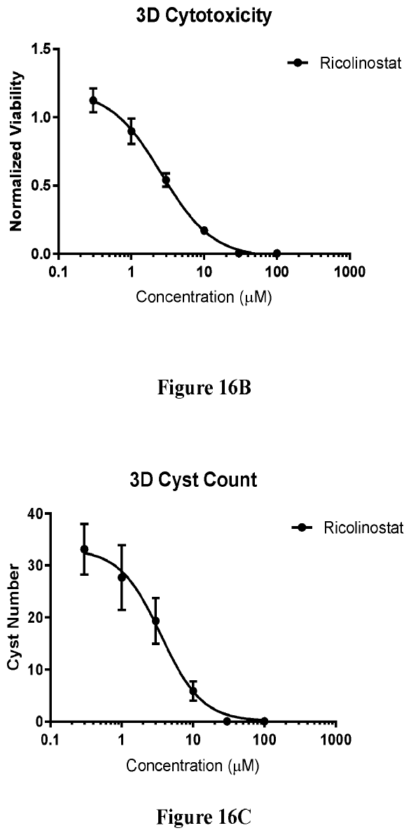

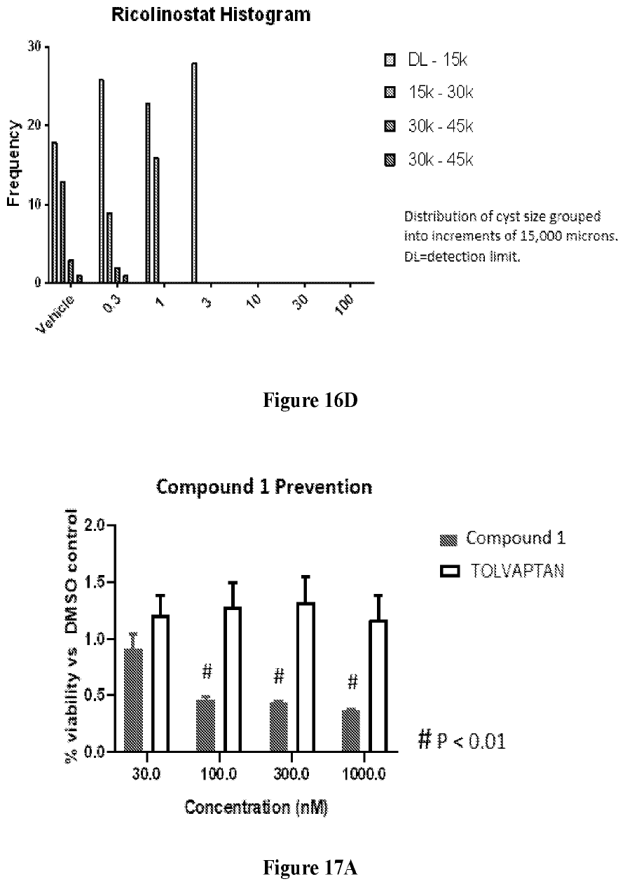

High-content imaging, cell viability analysis, cyst counting, and cyst size analysis were performed on cultures in the presence of an industry standard control drug, ricolinostat (0.3-100 μM). Results are shown in FIGS. 16A-D, showing typical high-content images of cysts throughout the well, 3D Biogel cell viability versus cyst counts, and a histogram of cyst size analysis using artificial intelligence algorithm and “binning” method.

### Compound Screening in Prevention, Attack, and Reduction Assays

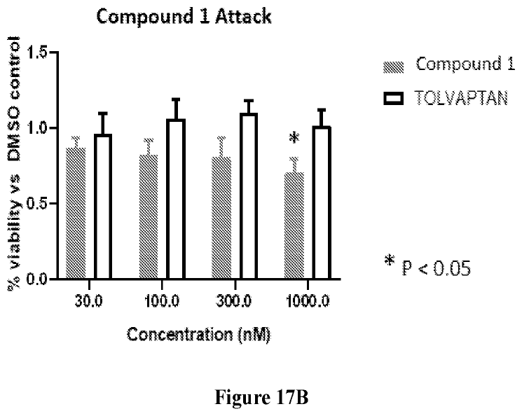

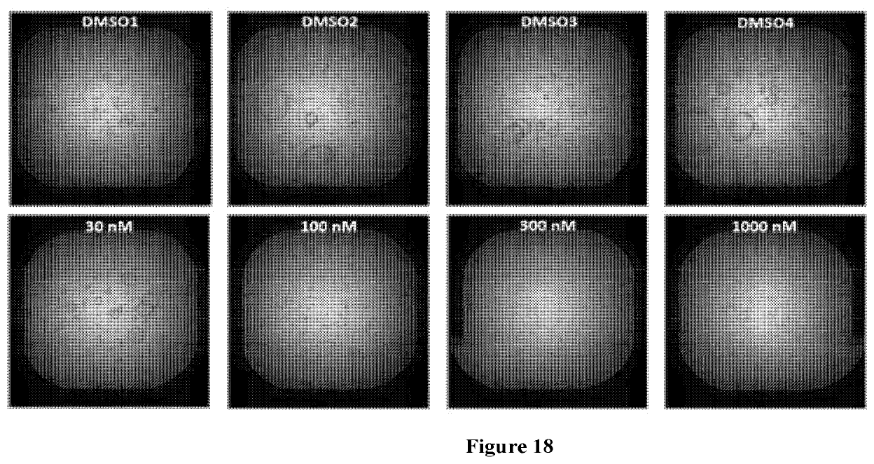

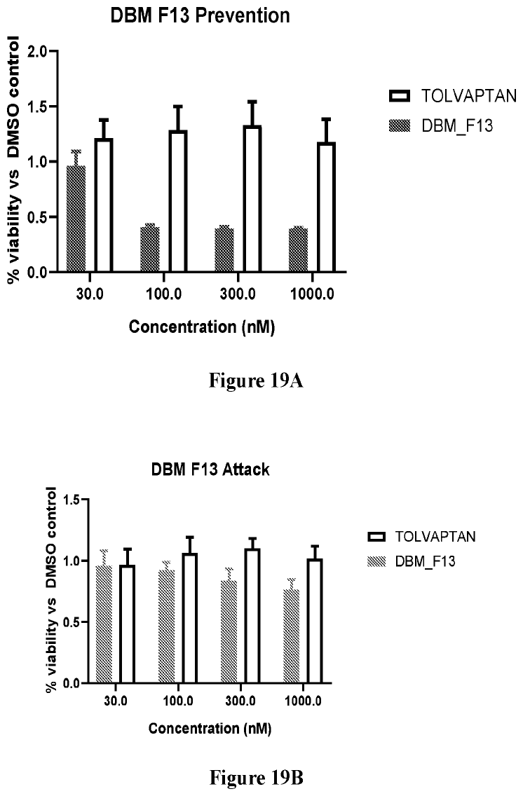

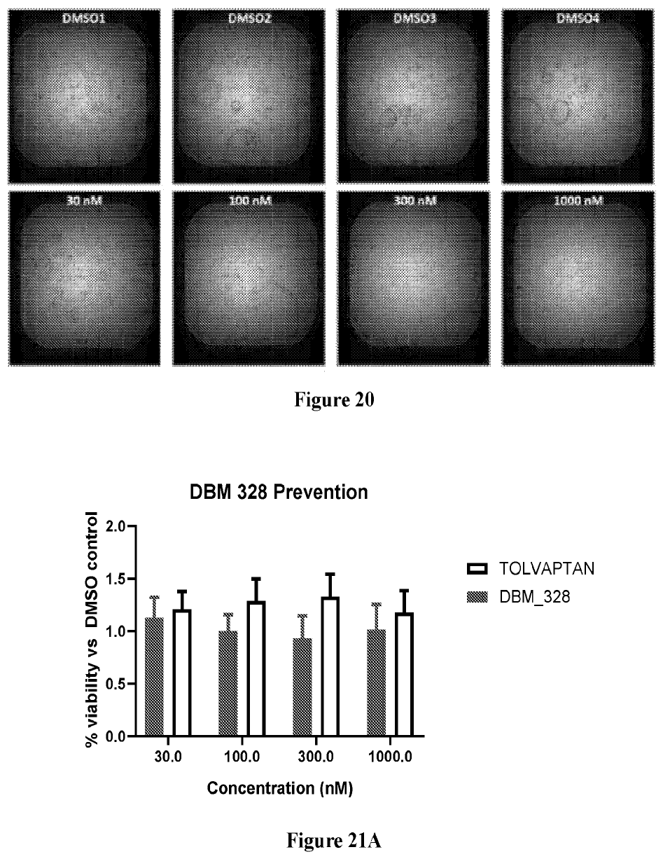

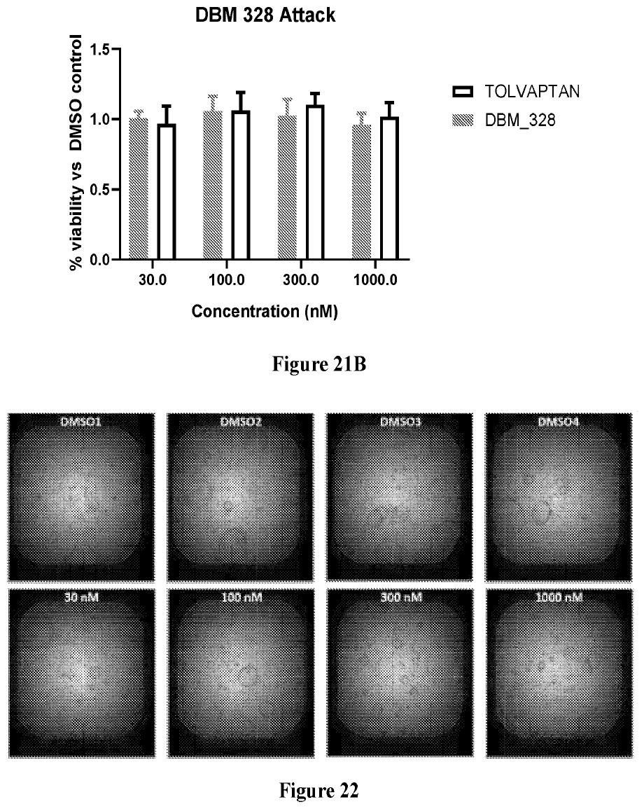

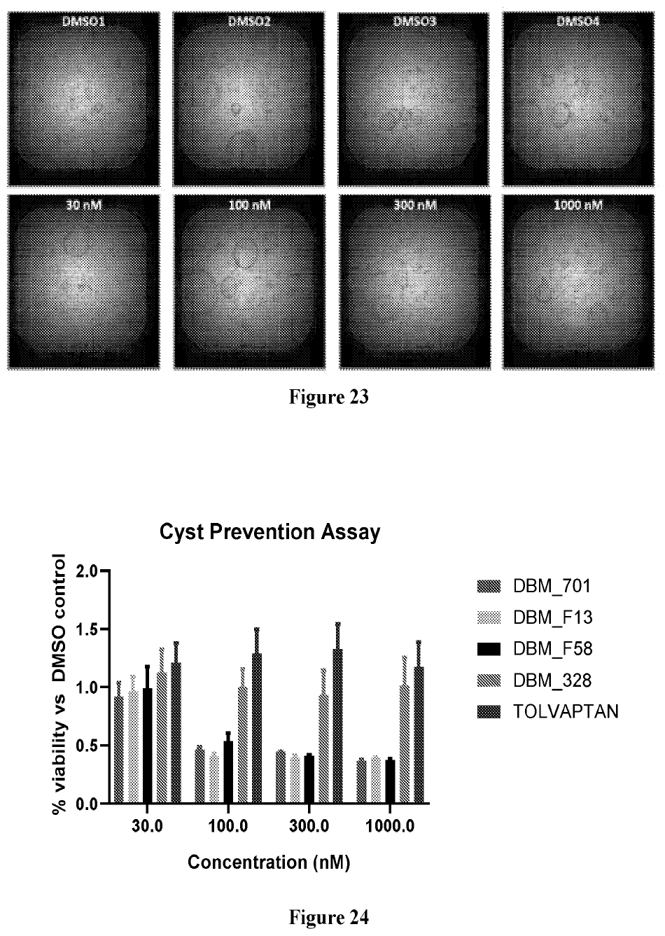

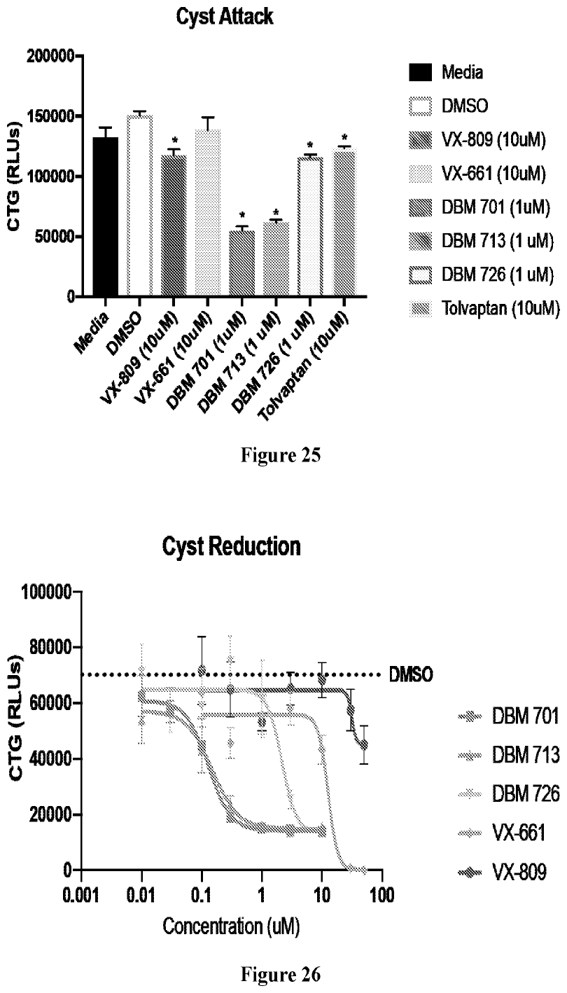

Compound 1 was tested in both prevention and attack assays. Results are shown in FIG. 17A and FIG. 17B comparing the results for Compound 1 (n=9) with those for tolvaptan (n=9). Concentrations of 30-1000 nM were tested for each, and data were expressed as percent viability versus DMSO control (n=12). Compound 1 had nanomolar efficacy, whereas tolvaptan was ineffective in the same ranges. Statistical analysis was performed using ANOVA. Cyst attenuation in cyst prevention assays was observed at concentrations of Compound 1 greater than 30 nM, as shown in FIG. 18. Compound 2 results in similar results in prevention and attack assays, as shown in FIGS. 19 and 20. However, Compound 3, an analog of Compound 1, was ineffective in both prevention and attack assays at the same concentrations, as shown in FIGS. 21 and 22. Tolvaptan was determined to be effective in cyst prevention assays only at mid-micromolar concentrations, as shown in FIG. 23. FIG. 24 shows the results for compounds 1, 2, 4, and 3 and tolvaptan at nanomolar concentrations. The IC50 for Compounds 1, 2.4, and 3 is 100 nM or lower, whereas the IC50 for tolvaptan is greater than 10 μM. Tolvaptan is only slightly more effective in cAMP stimulated assays.

Compounds 1, 2, and 5 were also compared to Vertex CFTR corrector drugs (10 μM) and to Tolvaptan (10 μM) on primary human ADPKD cultures with multiple enlarged cysts per 3D Biogel in a 3D CellTiterGLO Assay of cell viability. Cultures were incubated for 2 weeks before 1-week treatment with the drugs or DMSO control and media only controls. With Compounds 1 and 2, 60-65% inhibition was observed, whereas Compound 6 (an analog of Compound 1), the Vertex CFTR corrector drugs, and Tolvaptan only showed about 20% inhibition at a 10-fold higher concentration.

Compounds 1, 2, and 6 were compared to Vertex CFTR corrector drugs in a 3D CellTiterGLO assay of cell viability using a CRC design in which ADPKD cell cultures had a few small cysts per 3D Biogel starting to form (i.e., incubated 10 days before 1 week of treatment with drugs or media or DMSO only). Results are shown in FIG. 26 for a cyst reduction assay. Compounds 1 and 2 had similar CRC relationships, with an IC50 of 200 nM and an inhibition of 75% that reflects cytostasis or growth arrest. Compound 6 was much less potent with an IC50 of 3 μM, but with a similar maximum inhibition. In contrast, the VX-661 showed an IC50 of 30 μM and a complete inhibition to no signal, indicative of overt cytotoxicity. VX-809 showed only a partial effect and only at the highest concentrations, similar to tolvaptan.

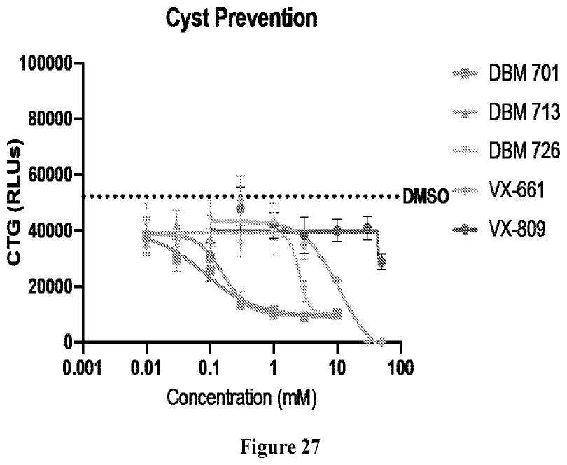

Similarly, FIG. 27 shows results for a cyst prevention assay using Compounds 1, 2, and 6 and Vertex CFTR corrector drugs in a 3D CellTiterGLO assay of cell viability using a CRC design in which ADPKD cell cultures. In this assay, the ADPKD cell cultures were incubated on 4 days before 1 week of treatment with drugs, media only or DMSO only. Compound 1 showed an IC50 of less than 100 nM, and Compound 2 showed an IC50 of less than 200 nM. An inhibition of 75% was observed again, reflecting cytostatis or growth arrest. Compound 6 was much less potent, with an IC50 of 4 μM but with a similar maximum inhibition. By contrast VX-661 showed an IC50 of 10 μM and a complete inhibition to no signal, indicating overt cytotoxicity. VX-809 showed only a partial effect and only at the highest concentration.

### Compound Screening of Normal Human Kidney (NHK) Cells

An initial screen of small molecule collections on normal human kidney (NHK) cells grown in 3D Biogels is performed to insure that these molecules are not toxic to NHK cells and that they do not disrupt tubulogenesis in 3D Biogels. Small molecules that are not harmful are assessed in the primary human ADPKD cystogenesis assays described above, to determine if they attenuate cystogenesis over time. Supernatants are collected at study end to determine if production/secretion of select inflammatory mediators is attenuated (so called potential inhibitors of the ADPKD inflammasome). Validated inhibitors of ADPKD cystogenesis and the inflammasome are tested in cells derived from at least 2 additional ADPKD donors.

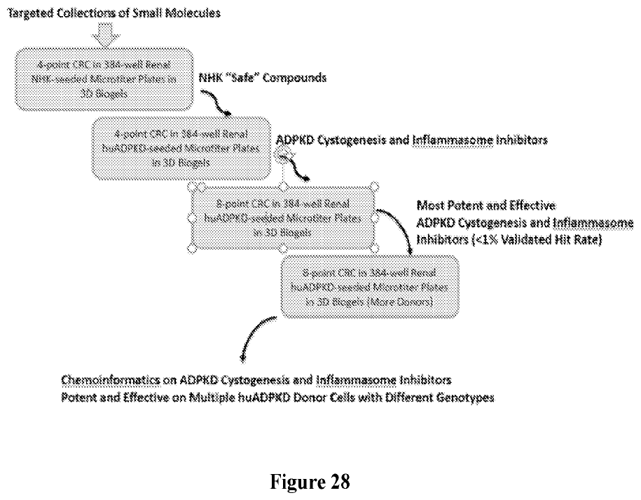

FIG. 28 is a schematic of a process for identifying ADPKD cystogenic inhibitors using the assays described herein. Briefly, therapeutic candidates are initially be screened on primary normal human kidney cell platforms to insure lack of cytotoxicity so as to identify “safe” compounds to screen as human ADPKD cystogenesis inhibitors and/or inflammasome inhibitors. A multiplexed assay is used where high-content imaging documents attenuation of cyst formation or shrinkage of cysts, while the media supernatant overlaying the 3D Biogels is harvested to quantify chemokines, cytokines and other biomarkers of the human ADPKD “inflammasome” by AlphaLISA technology.

1. A method of producing immortalized, cystogenic, single cyst-derived cells from a polycystic kidney comprising
(a) isolating one or more cells from a single cyst in a polycystic kidney;
(b) culturing the isolated one or more cells in a culture dish or well under conditions for expanding the one or more cells to create a plurality of single cyst-derived cells;
(c) transferring the plurality of single-cyst derived cells to a three dimensional culture system configured for cyst formation;
(d) determining whether the plurality of single-cyst derived cells form cyst-like structures in the three dimensional culture; and
(e) immortalizing one or more of the plurality of single-cyst derived cells that form cyst-like structures.

2. The method of claim 1, further comprising genotyping or whole exome sequencing the one or more isolated cells.

3. The method of claim 1, wherein the transferred plurality of single-cyst derived cells have been passaged fewer than 5 times prior to transfer.

4. The method of claim 3, wherein the transferred plurality of cells has been passaged fewer than 3 times.

5. The method of claim 1, wherein the isolated one or more cells are cultured on a permeable filter.

6. The method of claim 1, wherein the immortalization is performed using a telomerase.

7. The method of claim 1, wherein the immortalization is performed using a viral oncogene.

8. The method of claim 7, wherein the viral oncogene is SV40.

9. A primary culture of cystogenic, single cyst-derived cells from a polycystic kidney.

10. The primary culture of claim 9, wherein the cells are produced by
(a) isolating one or more cells from a single cyst in a polycystic kidney;
(b) culturing the isolated one or more cells in a culture dish or well under conditions for expanding the one or more cells to create a plurality of single cyst-derived cells;
(c) transferring a first subset of the plurality of single-cyst derived cells to a three dimensional culture system configured for cyst formation;
(d) determining the first subset of the plurality of single-cyst derived cells form cyst-like structures in the three dimensional culture; and
(e) isolating a second subset of the plurality of single cyst-derived cells to create a primary culture of cystogenic, single cyst-derived cells from a polycystic kidney.

11. (canceled)

12. The population of immortalized, cystogenic, single cyst-derived cells from a polycystic kidney derived by the method of claim 1.

13. A three dimensional culture of immortalized, cystogenic, single cyst-derived cells from a polycystic kidney comprising
(a) the population of immortalized, cystogenic, single cyst-derived cells of claim 11 and
(b) a three dimensional gel.

14. A method of screening for an agent that prevents or treats polycystic kidney disease comprising
(a) contacting a culture of cystogenic, single cyst-derived cells from a polycystic kidney with an agent to be screened and
(b) transferring the contacted cells to a three dimensional culture system configured for cyst formation; and
(c) determining the level of cyst-like formations in the three dimensional culture system of the contacted cells after treatment with the agent as compared to a control level, a reduced level of cyst-like formations indicating the agent prevents or treats polycystic kidney disease.

15. The method of claim 14, wherein the culture of cystogenic, single cyst-derived cells from a polycystic kidney comprises a primary culture of cells isolated from a single cyst or immortalized cells.

16. The method of claim 14, further comprising correlating the agent that prevents or treats polycystic kidney disease with a specific genetic profile of the cystogenic, single cyst-derived cells.

17. The method of claim 14, wherein the control level is the cystogenic level of untreated cells derived from the same cyst.

18. A method of screening for an agent that prevents or treats polycystic kidney disease comprising
(a) contacting a three dimensional culture system of cystogenic, single cyst-derived cells from a polycystic kidney with an agent to be screened and
(b) determining the level of cyst-like formations in the three dimensional culture system of the contacted cells after treatment with the agent as compared to a control level, a reduced level of cystogenic capacity indicating the agent prevents or treats polycystic kidney disease.

19. The method of claim 18, wherein the culture of cystogenic, single cyst-derived cells from a polycystic kidney comprises a primary culture of cells isolated from a single cyst or immortalized cells.

20. The method of claim 18, further comprising correlating the agent that prevents or treats polycystic kidney disease with a specific genetic profile of the cystogenic, single cyst-derived cells.

21. The method of claim 18, wherein the control level is the cystogenic level of untreated cells derived from the same cyst.
